(19) United States
(12) Patent Application Publication
(10) Pub. No.: US 2007/0129764 A1
(43) Pub. Date: Jun. 7, 2007

(54) Method and apparatus for optimizing pacing parameters

(76) Inventor: John E. Burnes, Andover, MN (US)

Correspondence Address: MEDTRONIC, INC., 710 MEDTRONIC PARK, MINNEAPOLIS, MN, 55432-9924, US

(21) Appl. No.: 11/295,272

(22) Filed: Dec. 6, 2005

(51) Int. Cl. A61N 1/365

(52) U.S. Cl. 607/18; 607/23

(57) ABSTRACT

A program for optimizing a pacing interval in an implantable medical device. The program includes setting the pacing interval to a baseline and measuring a physiologic sensor variable. The program measures the transient change in the physiologic sensor variable each time the pacing interval is changed. The pacing interval setting associated with the maximum transient change is deemed to be the optimal setting.

### BACKGROUND

Cardiac resynchronization therapy (CRT) has been clinically demonstrated to improve indices of cardiac function in patients suffering from congestive heart failure. CRT involves cardiac pacing that may be applied to one or both ventricles or multiple heart chambers, including one or both atria, to improve cardiac chamber coordination, which in turn is thought to improve pumping efficiency and stroke volume and pumping efficiency. Follow-up of patients undergoing CRT has shown improvements in clinical indices as well as hemodynamic measures of cardiac function, left ventricular volumes, and wall motion.

Optimization of pacing parameters and intervals is important to maximizing heart function. However, physicians are challenged in selecting the optimal pacing intervals, such as the intervals between the atria and ventricles (AV intervals) and between the ventricles (VV intervals). Selection of pacing intervals may be based on echocardiographic evaluation of cardiac function or a variety of other selection methods that attempt to optimize cardiac function or hemodynamic status.

### SUMMARY

The present disclosure and associated embodiments are directed to the optimization of a pacing interval in an implantable medical device. Instructions for performing the optimization can be programmed onto a computer-readable medium. Optimization comprises setting a pacing interval to a baseline, measuring a baseline physiologic sensor variable, and measuring the transient change in the sensor variable in response to changes in the pacing interval. The change in pacing interval can be performed two or more times. The pacing interval which produces the maximum transient change in the sensor variable can be considered optimal or a candidate valued for later regarding testing and/or other confirmatory evaluation. In some embodiments, this optimal pacing interval can be adjusted by a physiologic offset interval.

The transient change in the value of the physiologic sensor variable in response to a change in the pacing interval can be measured at each pacing interval setting. The maximum transient change of the measured transient changes can be identified using a variety of methods. In some embodiments, the maximum transient change is determined by fitting a curve to the measured transient changes to determine the point on the curve where the change is maximum.

The pacing interval may be adjusted randomly or systematically. In some embodiments, the pacing interval can be adjusted and the transient change in the physiologic sensor variable can be measured using an iterative process. For example, the change in the pacing interval can be based upon a measured transient change in a sensor value at a previous pacing interval.

### BRIEF DESCRIPTION OF THE DRAWINGS

The following drawings are illustrative of particular embodiments of the present invention and therefore do not limit the scope of the invention. The drawings are intended for use in conjunction with the explanations in the following detailed description. Embodiments of the present invention will hereinafter be described in conjunction with the appended drawings, wherein like numerals denote like elements.

FIG. 1 is an example of an implantable medical device.

FIG. 2 is a schematic diagram of an exemplary multi-chamber monitoring monitor/sensor.

FIG. 3 is a schematic diagram of a pacing, sensing and parameter measuring channel.

FIG. 4 is a block diagram of a method of optimizing a pacing interval according to some embodiments.

FIG. 5 is a diagram of a transient increase in a sensed variable in response to a change in a pacing interval.

FIG. 6a is laboratory data showing the change in LV flow in response to various AV intervals.

FIG. 6b is laboratory data showing the change in arterial pressure in response to various AV intervals.

FIG. 6c is laboratory data showing the change in RV pulse pressure in response to various AV intervals.

FIG. 6d is laboratory data showing the change in LV pulse pressure in response to various AV intervals.

### DETAILED DESCRIPTION

The following detailed description is exemplary in nature and is not intended to limit the scope, applicability, or configuration of the invention in any way. Rather, the following description provides practical illustrations for implementing exemplary embodiments of the present invention.

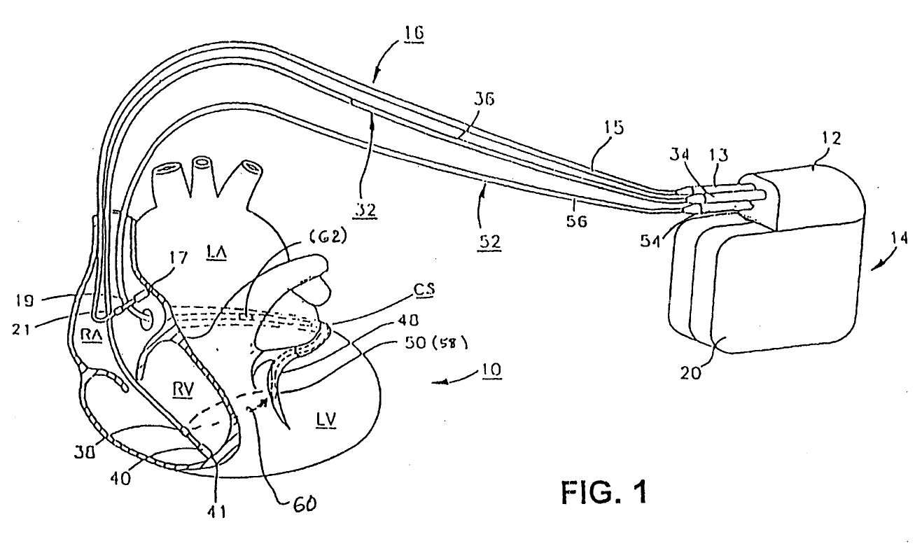

FIG. 1 is a schematic representation of an implantable medical device (IMD) 14 that may be used in accordance with certain embodiments of the invention. The IMD 14 may be any device that is capable of measuring hemodynamic parameters (e.g., blood pressure signals) from within a ventricle of a patient's heart, and which may further be capable of measuring other signals, such as the patient's electrogram (EGM).

In FIG. 1, heart 10 includes the right atrium (RA), left atrium (LA), right ventricle (RV), left ventricle (LV), and the coronary sinus (CS) extending from the opening in the right atrium laterally around the atria to form the great vein.

FIG. 1 depicts IMD 14 in relation to heart 10. In certain embodiments, IMD 14 may be an implantable, multi-channel cardiac pacemaker that may be used for restoring AV synchronous contractions of the atrial and ventricular chambers and simultaneous or sequential pacing of the right and left ventricles. Three endocardial leads 16, 32 and 52 connect the IMD 14 with the RA, the RV and the LV, respectively. Each lead has at least one electrical conductor and pace/sense electrode, and a can electrode 20 may be formed as part of the outer surface of the housing of the IMD 14. The pace/sense electrodes and can electrode 20 may be selectively employed to provide a number of unipolar and bipolar pace/sense electrode combinations for pacing and sensing functions. The depicted positions in or about the right and left heart chambers are merely exemplary. Moreover other leads and pace/sense electrodes may be used instead of the depicted leads and pace/sense electrodes.

The IMD may also be an implantable cardioverter defibrillator (ICD), a CRT device, an implantable hemodynamic monitor (IHM), or other appropriate device or combination of such devices.

Typically, in pacing systems of the type illustrated in FIG. 1, the electrodes designated above as “pace/sense” electrodes are used for both pacing and sensing functions. In accordance with one aspect of the present invention, these “pace/sense” electrodes can be selected to be used exclusively as pace or sense electrodes or to be used in common as pace/sense electrodes in programmed combinations for sensing cardiac signals and delivering pace pulses along pacing and sensing vectors.

In addition, some or all of the leads shown in FIG. 1 could carry one or more pressure sensors for measuring blood pressures, and a series of spaced apart impedance sensing leads for recording volumetric measurements of the expansion and contraction of the RA, LA, RV and LV.

The leads and circuitry described above can be employed to record EGM signals, blood pressure signals, and impedance values over certain time intervals. The recorded data may be periodically telemetered out to a programmer operated by a physician or other healthcare worker in an uplink telemetry transmission during a telemetry session, for example.

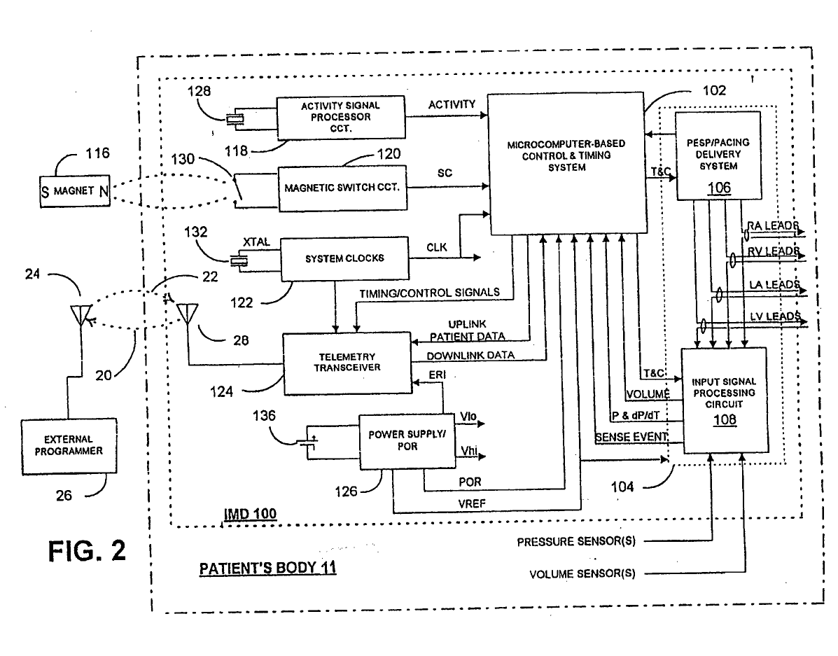

FIG. 2 depicts a system architecture of an exemplary multi-chamber monitor/sensor 100 implanted into a patient's body 11 that provides delivery of a therapy and/or physiologic input signal processing. The typical multi-chamber monitor/sensor 100 has a system architecture that is constructed about a microcomputer-based control and timing system 102 which varies in sophistication and complexity depending upon the type and functional features incorporated therein. The functions of microcomputer-based multi-chamber monitor/sensor control and timing system 102 are controlled by firmware and programmed software algorithms stored in RAM and ROM including PROM and EEPROM and are carried out using a CPU or ALU of a typical microprocessor core architecture. The microcomputer-based multi-chamber monitor/sensor control and timing system 102 may also include a watchdog circuit, a DMA controller, a block mover/reader, a CRC calculator, and other specific logic circuitry coupled together by on-chip data bus, address bus, power, clock, and control signal lines in paths or trees in a manner well known in the art. It will also be understood that control and timing of multi-chamber monitor/sensor 100 can be accomplished with dedicated circuit hardware or state machine logic rather than a programmed micro-computer.

The therapy delivery system 106 can be configured to include circuitry for delivering cardioversion/defibrillation shocks and/or cardiac pacing pulses delivered to the heart or cardiomyostimulation to a skeletal muscle wrapped about the heart. Alternately, the therapy delivery system 106 can be configured as a drug pump for delivering drugs into the heart to alleviate heart failure or to operate an implantable heart assist device or pump implanted in patients awaiting a heart transplant operation.

The input signal processing circuit 108 includes at least one physiologic sensor signal processing channel for sensing and processing a sensor derived signal from a physiologic sensor located in relation to a heart chamber or elsewhere in the body.

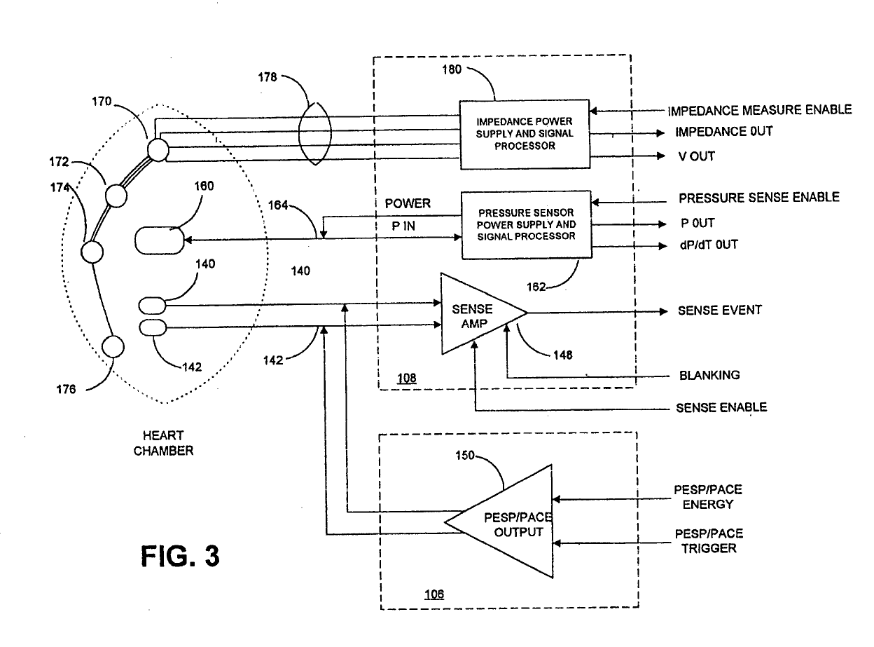

FIG. 3 schematically illustrates one pacing, sensing and parameter measuring channel in relation to one heart chamber. A pair of pace/sense electrodes 140,142, a pressure sensor 160, and a plurality, e.g., four, impedance measuring electrodes 170, 172, 174, 176 are located in operative relation to the heart 10.

The pair of pace/sense electrodes 140,142 are located in operative relation to the heart 10 and coupled through lead conductors 144 and 146, respectively, to the inputs of a sense amplifier 148 located within the input signal processing circuit 108. The sense amplifier 148 is selectively enabled by the presence of a sense enable signal that is provided by control and timing system 102. The sense amplifier 148 is enabled during prescribed times when pacing is either enabled or not enabled in a manner known in the pacing art. The blanking signal is provided by control and timing system 102 upon delivery of a pacing pulse or pulse train to disconnect the sense amplifier inputs from the lead conductors 144 and 146 for a short blanking period in a manner well known in the art. The sense amplifier provides a sense event signal signifying the contraction of the heart chamber commencing a heart cycle based upon characteristics of the EGM. The control and timing system responds to non-refractory sense events by restarting an escape interval (EI) timer timing out the EI for the heart chamber, in a manner well known in the pacing art.

The pressure sensor 160 is coupled to a pressure sensor power supply and signal processor 162 within the input signal processing circuit 108 through a set of lead conductors 164. Lead conductors 164 convey power to the pressure sensor 160, and convey sampled blood pressure signals from the pressure sensor 160 to the pressure sensor power supply and signal processor 162. The pressure sensor power supply and signal processor 162 samples the blood pressure impinging upon a transducer surface of the sensor 160 located within the heart chamber when enabled by a pressure sense enable signal from the control and timing system 102. Absolute pressure (P), developed pressure (DP) and pressure rate of change (dP/dt) sample values can be developed by the pressure sensor power supply and signal processor 162 or by the control and timing system 102 for storage and processing.

A variety of hemodynamic parameters may be recorded, for example, including right ventricular (RV) systolic and diastolic pressures (RVSP and RVDP), estimated pulmonary artery diastolic pressure (ePAD), pressure changes with respect to time (dP/dt), heart rate, activity, and temperature. Some parameters may be derived from others, rather than being directly measured. For example, the ePAD parameter may be derived from RV pressures at the moment of pulmonary valve opening, and heart rate may be derived from information in an intracardiac electrogram (EGM) recording.

The set of impedance electrodes 170, 172, 174 and 176 is coupled by a set of conductors 178 and is formed as a lead that is coupled to the impedance power supply and signal processor 180. Impedance-based measurements of cardiac parameters such as stroke volume are known in the art, such as an impedance lead having plural pairs of spaced surface electrodes located within the heart 10. The spaced apart electrodes can also be disposed along impedance leads lodged in cardiac vessels, e.g., the coronary sinus and great vein or attached to the epicardium around the heart chamber. The impedance lead may be combined with the pace/sense and/or pressure sensor bearing lead.

The data stored by IMD 14 may include continuous monitoring of various parameters, for example recording intracardiac EGM data at sampling rates as fast as 256 Hz or faster. In certain embodiments of the invention, an IHM may alternately store summary forms of data that may allow storage of data representing longer periods of time. In one embodiment, hemodynamic pressure parameters may be summarized by storing a number of representative values that describe the hemodynamic parameter over a given storage interval. The mean, median, an upper percentile, and a lower percentile are examples of representative values that may be stored by an IHM to summarize data over an interval of time (e.g., the storage interval).

Hemodynamic parameters that may be used in accordance with various embodiments of the invention include parameters that are directly measured, such as RVDP and RVSP, as well as parameters that may be derived from other pressure parameters, such as estimated pulmonary artery diastolic pressure (ePAD), rate of pressure change (dP/dt), etc.

A cardiac resynchronization therapy (CRT) device is provided to a patient's heart using baseline pacing or nominal intervals. However, these pacing intervals may not be optimal. Furthermore, different pacing intervals may be optimal under different circumstances, such as at rest versus during exertion.

Thus it is desirable for the device to be capable of adjusting the pacing intervals to optimize cardiac function. Optimization of cardiac function is particularly important in patients whose cardiac function is impaired, as is the case with many recipients of cardiac resynchronization devices.

Certain embodiments of the present invention optimize a pacing interval (PI) by varying the interval and observing the transient change in a sensed variable (ΔSV) as compared to a baseline sensed variable (SV) value. A computer readable medium may be programmed to perform the optimization.

The change in pacing interval and the measurement of the change in the sensed variable may be repeated until the pacing interval which produces the maximum transient change in the sensed variable is identified. It is believed that this pacing interval represents an optimal pacing interval.

Appropriate pacing intervals which may be optimized according to embodiments of this invention include the AV interval, including right atrium to right ventricle as well as right atrium to left ventricle interval, the VV interval (the interval ventricular paces), and the pre-excitation interval (PEI). Atrial activation may be intrinsic or may be stimulated by the resynchronization device during optimization. The resynchronization device could provide single, dual, triple or quadruple chamber pacing. Alternatively, it could provide pacing outside of the heart such as by an epicardial patch or other device, or through a combination of endocardial and extra-cardiac stimulation. It is preferred that the device includes a sensor such as an implanted sensor that is capable of measuring the variable of interest. For example, a cardiac resynchronization device providing three chamber pacing and having a sensor for measuring right ventricular (RV) pressure is appropriate for some embodiments. The sensed variable is a physiological variable related to heart function. The sensed variable may be an autonomically controlled parameter. Examples of autonomically controlled parameters include pressure, cardiac output and cardiac flow. Appropriate sensed variables include, for example, RV, LV or LA pressure derived parameters, such as dP/dt (change over time), dP/dtₘₐₓ, dP/dtₘᵢₙ, end systolic pressure, end diastolic pressure, pulse pressure, and minimum and maximum pressures. Other variables include arterial pressure, myocardial acceleration, impedance, flow and tensiometric measurements. Alternatively, peripheral measurements including variables such as pulse pressure or oxygenation level may be evaluated. Although a sensed variable such as RV pressure may vary throughout the cycle of the heart beat, a measure of the variable, such as its peak or an average during a single cycle, may be selected to represent as the value of the variable for a particular beat.

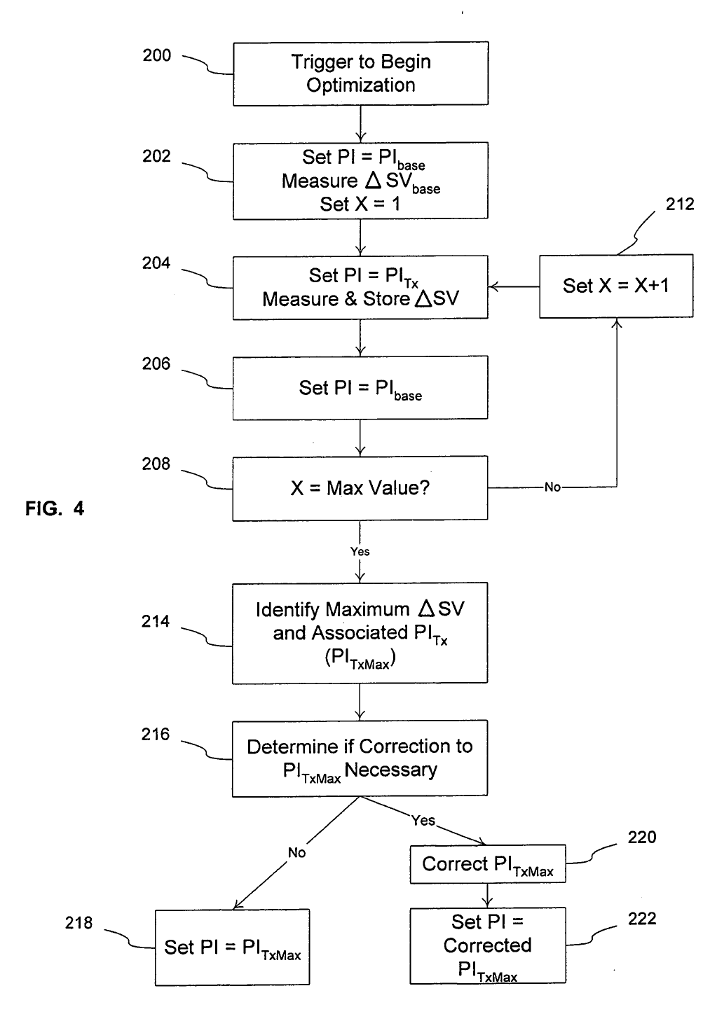

An example of a process of optimization of a pacing interval is illustrated in FIG. 4. The first step in the process, step 200, is triggering of the optimization. The initiation of pacing interval optimization by a device may be automatic or manual. For example, the device may automatically optimize the pacing interval every night or after a certain amount of time has passed since implantation or the last optimization. Alternatively, the device may automatically initiate optimization when it senses a change in physiologic state. Examples of possible physiological changes that might trigger optimization include suspected heart failure decompensation, increased heart rate, sleeping or exertion. For example, the device may trigger optimization when it detects a heart rate of approximately 10 to 40 beats per minute above the resting heart rate. In another embodiment, optimization could be triggered by programmer or physician initiation in addition to, or as an alternative to, one or more methods of automatic initiation.

After initiation is triggered, the optimization process begins at step 202 in FIG. 4 by measuring a baseline value for the sensed variable (SV_{base}) with the device set to a baseline pacing interval (PI_{base}) while at an elevated heart rate. The value of SV_{base} can be determined by measuring the sensed variable over an number of beats. For example, SV_{base} may be the average of SV over a number of beats at the baseline PI. The baseline pacing interval is the pacing interval at the start of a test, to which the transient response to test pacing intervals is compared. It may be the pacing interval with which the device was functioning prior to initiation of the optimization test. The baseline pacing interval may be an estimate of the optimal pacing interval manually supplied by the clinician via a programmer or automatically estimated by the device. It may also be an interval that has previously been estimated to be optimal based on echocardiographic data, by previous optimization processes according to embodiments of this invention, or by other forms of optimization.

Once an adequate baseline sensed variable has been measured and the underlying rhythm is considered stable, the device changes the pacing interval from the baseline interval (PI_{base}) to a new test interval (PI_{T1}) at step 204 in FIG. 4. The new pacing interval may be either increased or decreased compared to the baseline (PI_{base}). Also, the amount and direction of change in the pacing interval may either be systematic or random. For example, in some embodiments, the pacing interval to be optimized is the AV interval and the sensed variable is the RV pressure. The AV interval may be changed from AV_{base} to AV_{T1}, which may be longer or shorter than AV_{base}. In this example, the transient change in RV pressure (ΔRV) in response to AV_{T1} may then be measured.

The sensed variable may undergo a transient change, such as an increase, immediately following the change in the pacing interval. This increase occurs due to improved cardiac function in response to the new pacing interval. While not intending to be bound by theory, it is believed that the increase is transient, reflecting the inherent ability of the body's autonomic system to adjust to an increase in cardiac output beyond what the body needs at that moment, resulting from the changed pacing interval. Thus the measurement of this transient change with various pacing intervals allows identification of the pacing interval resulting in maximal functionality of the heart (the optimal pacing interval).

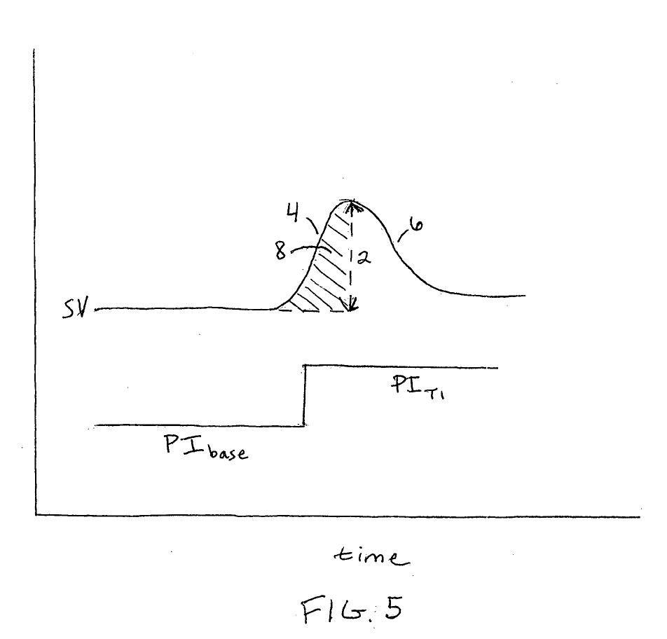

The transient change in the sensed variable is detected by the device sensor in step 204 of FIG. 4. An example of a transient change in a sensed variable in response to a change in the pacing interval is graphically represented in FIG. 5. The figure represents the value of the SV for a number of beats before and after a change in a pacing interval. Although the measured data for the SV would actually be a number of discrete values, with one point for each heart beat, the data could be used to generate a continuous curve as shown in FIG. 5. The heart is assumed to be in a steady state at the end of the baseline time period, so the sensed variable, SV, is presumed constant over several cardiac cycles paced at the baseline interval, PI_{base}. When the pacing interval is changed to the test pacing interval PI_{T1} over several cardiac cycles, there is a transient increase in the SV, which then falls to a new baseline, which may or may not be equal to the previous baseline. The value which is taken to be the ΔSV may be the maximum increase 2 over the baseline SV. Alternatively, ΔSV may be, for example, the slope of the initial deflection from the baseline 4 (in this case a transient increase), the slope of the return to baseline 6 (in this case a transient decrease), or the integrated area under a portion 8 or all of the region transient change. While this example shows a transient increase of the sensed variable over the baseline, indicating that the test pacing interval is superior to the baseline pacing interval, it is possible that the sensed variable will transiently decrease. A transient decrease would indicate that the changed pacing interval is inferior to the baseline pacing interval. A lack of change in the sensed variable would indicate that the baseline and test pacing intervals are substantially equivalent.

In one embodiment, the device is returned to the baseline pacing interval in step 206 following the adjustment in the pacing interval. After the sensed variable returns to the baseline level, the device again adjusts the pacing interval to another new value in step 204 and the transient change in the sensed variable is again measured. For example, AV_{base} may be adjusted to AV_{T1} and the ΔRV may be determined. The device may then be returned to AV_{base} such that the RV returns to baseline. The AV interval may then be adjusted again to a new AV interval, AV_{T2}, and the ΔRV may be determined at AV_{T2}. The device may then return to the baseline AV interval and may repeat the process for a third or more consecutive AV test intervals.

This iterative process of adjusting the pacing interval may be repeated until an optimal pacing interval is determined in step 214 based on the change in the sensed variable. Thus when the sensed variable changes by a maximum amount, the pacing interval is optimal. The maximum change may be determined, for example, by plotting the change in the sensed variable versus the pacing interval and curve fitting the plot to determine the point on the curve where the change in the sensed variable is maximal. The pacing interval which corresponds to this point is the optimum pacing interval. Alternatively, an algorithm may be used to identify the maximum ΔSV and the PI which corresponds to the maximum ΔSV.

The magnitude and direction of change of each consecutive test pacing interval could be random or could follow a pattern. For example, when a randomly selected pacing interval is used, each test interval could be larger or smaller than the baseline interval and could selected randomly, with data regarding the sensed variable collected by the device for each test pacing interval. Alternatively, the pacing interval could be stepwise increased or decreased. For example, the device could first test with a shorter pacing interval than the baseline. If the sensed variable shows a transient decrease or no transient increase, the pacing interval could be adjusted in the opposite direction to be lengthened relative to the baseline interval. If the sensed variable shows a transient increase relative to the baseline, the pacing interval could be further increased. The change in the sensed variable at this second test interval could be compared to the change in sensed variable at the first test interval, and if the change in the variable was greater at the second test pacing interval, the pacing interval could be lengthened further. This step wise process could continue until the sensed variable no longer increased or decreased relative to the preceding pacing interval. At this point, the pacing interval which produces the greatest change in the sensed variable may be identified as the optimum. Optionally, this optimal pacing interval could be further refined by making smaller adjustments in the pacing interval and comparing the relative ΔSV until a more precise optimal pacing interval is identified.

In an alternative embodiment, the device may not return to the original baseline pacing interval after a particular test interval setting, but rather a new baseline may be set at the test pacing interval a certain period of time after the body has adjusted to the new pacing interval and a cardiovascular or cardiac steady state has occurred. This period of time may be approximately 10 to 30 beats after the adjustment to the pacing interval. Under this embodiment, it is believed that the pacing interval should remain at each test interval a relatively greater number of cardiac cycles in order for the transient response to completely conclude. By holding the pacing interval at a particular test interval over a greater number of cardiac cycles, the test interval, in effect, becomes the new baseline. The pacing interval is then adjusted again and the transient change in the sensed variable is measured at this third pacing interval. The device may repeat this process such that the pacing interval is adjusted and the transient change in the sensed variable is measured without returning to the original baseline pacing interval.

In one embodiment, the device could first test with a shorter pacing interval (PI_{T1}) than the current baseline pacing interval (PI_{base1}). If the sensed variable shows a transient decrease or no transient increase, the current pacing interval could be returned to the original pacing interval for a period of time sufficient to reestablish the original baseline (PI_{base1}). The pacing interval could then be adjusted in the opposite direction to be lengthened relative to the baseline interval (PI_{T2}). If the sensed variable shows a transient increase relative to the baseline, the current pacing interval (PI_{T2}) would be maintained long enough to establish it as a new baseline (PI_{base2}), and the pacing interval could be further increased (PI_{T3}). If the sensed variable shows a transient decrease or no transient increase, the current pacing interval could be returned to the previous pacing interval (PI_{base2} which is equal to PI_{T2}), which could be interpreted as the optimal PI. If the sensed variable shows a transient increase relative to the baseline (PI_{base2}), the current pacing interval (PI_{T2}) would be maintained long enough to establish it as a new baseline (PI_{base3}), and the pacing interval could be iterated out in such a fashion until no change or a negative change is observed.

Pacing interval optimization may be performed with the heart rate at a resting or at an elevated rate, although transient changes in the sensed variable may be most easily detected at elevated heart rates. However, whether the heart rate is at a resting or an elevated rate, it should be held constant throughout the optimization process, as should all other parameters that are not being evaluated. For instance, if the pacing interval of interest is the AV interval, parameters such as the VV interval and pacing mode should be held constant. When optimization is performed at an elevated heart rate, the heart may be working harder or less efficiently to meet increased demand by the body. Therefore, while a pacing interval which is less than optimal may be sufficient at rest, an optimal pacing interval which allows the heart to function at maximum efficiency, may be more critical at higher heart rates. Thus, the optimum interval identified at an elevated heart rate may then be applied during heart rate elevation as well as at rest, when optimization is less necessary. Alternatively, the pacing interval identified at a particular elevated heart rate could be used to extrapolate appropriate intervals for other heart rates. This could be accomplished by performing pacing interval optimization at more than one heart rate and using this data to extrapolate pacing intervals for all heart rates, such as through curve fitting. Alternatively, the data obtained from pacing interval optimization at just one heart rate could be used to estimate appropriate intervals at other heart rates based on an algorithm and/or on typical pacing interval profiles. Thus the data obtained from pacing interval optimization may be used to create a reference of optimal pacing intervals for different heart rates for an individual. These values may be collected, stored, and updated by the device over time for use as the patient's heart rate changes throughout the day.

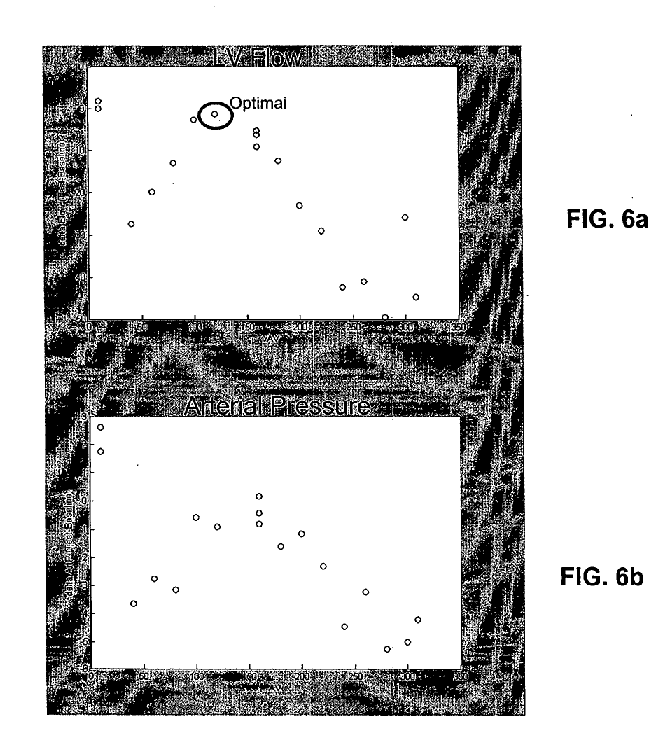

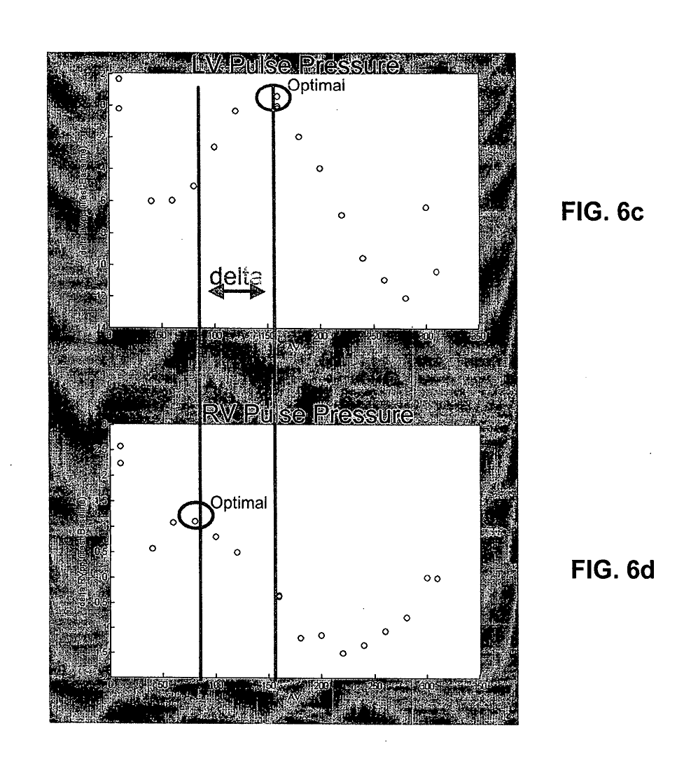

At times, repeated measurements of the sensed variable immediately after a change in the pacing interval may be required. This may be necessary, for example, when variations occur in the transient response of the sensed variable at test pacing intervals. As shown in FIG. 6b, for example, the transient change in arterial pressure in response to a change in the AV interval does not produce a smooth curve. Therefore it may be appropriate to perform repeat testing at particular AV intervals to obtain more definitive data. In addition, ectopic beats such as premature ventricular contractions (PVCs) and premature atrial contractions (PACs) may lead to aberrant measurements. Therefore data obtained in conjunction with the occurrence of PVCs or PACs during the optimization process may have to be disregarded. Measurements of sensed variables before and after the PVC or PAC may be disregarded and the testing process at that pacing interval may be repeated. However, the occurrence of an arrhythmic event or multiple PVCs or PACs during testing may stimulate the device to discontinue the pacing interval optimization process. Detection of such events may also signal the device to desist from the performance of future optimization testing for a certain period of time or until testing is enabled again, such as by a programmer.

Various corrections to the optimum pacing interval may be required for implementation in a patient. For example, in embodiments in which the AV interval is being optimized, a correction to the AV interval may be required to compensate for the difference in the speed of conduction between an intrinsic or sensed atrial contraction and a paced atrial contraction. Intrinsic contractions begin in the SA node and typically spread via Bachmann's bundle and other parts of the heart's specialized conduction system throughout the right and left atria. Conduction through the specialized conduction system is faster than conduction through the myocardium, which relies upon cell to cell conduction. In contrast to intrinsic depolarizations, paced atrial depolarizations must first pass through the relatively slower cell to cell conducting cardiac muscle to reach the SA node and the specialized conduction system or may bypass the specialized conduction system altogether. Because the electrical signal must follow this prolonged pathway in paced contractions, the conduction time is increased. This difference between the conduction time for sensed versus paced atrial contractions is the atrial sensed-paced conduction offset (AₛₚCO)and varies among individuals from approximately 30 milliseconds to 70 milliseconds and may be as much as approximately 100 milliseconds. Thus the difference in conduction time between sensed and paced atrial contractions may be significant. This offset can be periodically or automatically calculated by measuring and subtracting the time from an atrial sense to a ventricular sense (Aₛ−RVₛ) from the time from an atrial pace to a ventricular sense (Aₚ−RVₛ), such that the effect can be calculated on the basis of the following equation: AₛₚCO=(Aₚ−RVₛ)−(Aₛ−RVₛ).

When the AV interval is being optimized, and depending upon the method with which the optimal AV interval is being evaluated, a correction equal to the atrial sensed-paced conduction offset may be required. For example, in some embodiments, the heart rate is paced during optimization of the AV interval. In such cases, it may be appropriate to subtract the offset from the optimal AV interval to determine the optimal AV interval for sensed atrial contractions. In contrast, in embodiments in which the optimization is performed while the atrium is intrinsically driven, the optimum AV interval would require adjustment by the atrial sensed-paced conduction offset for instances when the device operates with a paced atrium. A determination of whether correction to the pacing interval PI is made in step 216 of FIG. 4. If a correction is deemed necessary, the PI is corrected in step 220 and the corrected interval is adopted as the pacing interval PI in step 222. If a correction is not deemed necessary in step 216, the optimal pacing interval PI is adopted as the pacing interval in step 218.

One or more other corrections to the optimal pacing interval may be required. These corrections, or physiologic offset intervals, may be based upon physiologically derived parameters. Examples of physiological offset intervals which may be used to adjust the pacing interval include interventricular mechanical delay, interventricular electrical delay, interventricular conduction time, inter-atrial conduction time, atrial sensed-paced conduction offset, and septal to posterior wall mechanical delay. In some embodiments, the physiologic offset interval may be used to optimize the pacing interval for a location different from a location where the physiologic sensor variable is measured. An example of the adjustment of a pacing interval using a physiological offset variable includes some embodiments in which the AV interval is optimized by identifying the AV interval that produces the maximum ΔRV pressure. However, it is the left ventricle which supplies blood to the body and which may be unable to provide adequate supply during periods of demand, particularly in heart failure patients. Thus it may be preferable to optimize the AV interval for left ventricular performance rather than for right ventricular performance. In many patients, there may be a time delay between right and left ventricular contractions, as indicated by a difference between, for example, dP/dtₘₐₓ, the time of maximum pressure or the time of minimum pressure for each ventricle. This delay can be measured as an interventricular mechanical delay. The delay may be a constant for an individual and may be patient specific. It may be determined by an echocardiogram or during catheterization. Thus, the optimal AV interval determined by measuring transient changes in RV pressure provides the optimum AV interval based on right ventricular performance parameters. However, to determine the optimal AV interval for left ventricular performance, the optimum AV interval determined using RV pressure may need to be adjusted by a value, which could be determined from a physiologic measurement such as inter-ventricular mechanical delay, inter-atrial mechanical delay or inter-atrial electrical delay. This delay constant could also be derived by looking at the difference in optimal AV intervals from curves generated using RV pressure and another systemic pressure or flow signal (for example LV pulse pressure, arterial pulse pressure, cardiac output, or aotic flow) measured in the catheterization lab or via non-invasive equipment. Once determined by any of the methods discussed above, this delay constant may be added to the optimal test interval determined by measuring the RV pressure.

FIGS. 6a -6d shows optimization data according to an embodiment. As shown in the figure, there are increases in left ventricular flow, atrial pressure, left ventricular pressure and right ventricular pressure as the AV interval is changed with respect to the baseline AV interval in this canine example. However, while the increase in RV pressure is the greatest with an AV interval of approximately 80 milliseconds, the increase in LV pressure parameters and flow is greatest with an AV interval of approximately 155 milliseconds. The time difference between maximum functioning of the right and left ventricles, identified in the figure as delta, is presumed to be equivalent to the delay constant discussed above. Thus, if data regarding the LV pressure or aortic flow was not available and only the RV pressure was being evaluated, the AV interval for optimization of LV function could be determined by adding delta to the optimal AV interval derived from the right ventricular pressure parameters. This corrected AV interval may then be set as the new base AV interval in step 222 in FIG. 4. For example, optimization of the AV interval using RV pressure, at a particular heart rate and with a VV interval of zero, may indicate that the optimal AV interval is 60 milliseconds. However, prior measurements may have demonstrated an interventricular mechanical delay of 100 milliseconds for this individual. The optimal AV interval for left ventricular performance might therefore be the sum of these values, 160 milliseconds. The cardiac resynchronization device could then reset itself to perform biventricular pacing with an AV interval of 160 milliseconds and a VV interval of zero.

Additionally, the optimal AV interval for overall cardiac performance may be identified using the RV pulse pressure curve as the AV delay producing an intermediate change in pulse pressure, such as half way between the greatest and the least right ventricular pulse pressure changes, indicating a compromise between optimal and worst RV performance.

In addition to optimizing pacing intervals, the optimization process described herein may be used to identify non-responders to cardiac resynchronization therapy or to identify optimal pacing site. For example, if no positive change in the sensed variable is observed with any change in pacing interval or pacing mode, the individual might not respond to resynchronization therapy. In response to such a scenario, the device may stop delivering the therapy until a re-evaluation of the response shows effective therapy, thus saving energy consumption while the device is not delivering an ineffective therapy. In another embodiment, the optimization data may be used to select a preferred pacing site such that the electrode may be relocated by moving the lead. Alternatively or additionally, the lead may contain multiple electrodes such as a tip electrode and one or more ring electrodes. The optimization process could be used to select which electrodes to use to provide cardiac stimulation. For example, rather than adjusting a pacing interval, the site optimization process could entail adjusting the electrode location and measuring the change in the sensed variable. Thus the change in the sensed variable could be compared for different electrode pacing sites or pacing electrode pairs while all other variable, including the heart rate and pacing intervals, were held constant. Alternatively, site optimization could be performed simultaneously with pacing interval optimization such that pacing site and pacing intervals are each iteratively changed to identify the optimal combination of pacing site and pacing intervals which produces the greatest change in sensed variable.

What is claimed is:

1. A computer-readable medium programmed with instructions for performing a method of optimization of a pacing interval in an implantable medical device, the medium comprising instructions for causing a programmable processor to:
set the pacing interval to a baseline; measure a value of a baseline physiologic sensor variable at the baseline pacing interval;
change the pacing interval setting two or more times;
measure the transient change in the value of the physiologic sensor variable in response to the change in the pacing interval setting at each pacing interval setting;
determine a maximum transient change of the measured transient changes;
identify the pacing interval associated with the maximum transient change in the sensor variable as an optimal pacing interval; and
adjust the optimal pacing interval by at least one physiological offset interval.

2. A medium according to claim 1, wherein the physiologic offset interval defines a constant and relates to a patient specific measure of a physiologic interval.

3. A medium according to claim 1, wherein the physiologic offset interval includes one of interventricular mechanical delay, interventricular electrical delay, interventricular conduction time, inter-atrial conduction time, atrial sensed-paced conduction offset, or septal to posterior wall mechanical delay.

4. A medium according to claim 1, wherein the physiologic offset interval optimizes the pacing interval for a location different from a location where the physiologic sensor variable is measured.

5. A medium according to claim 4, wherein the physiologic sensor variable includes a measurement in a right ventricle and the physiologic offset interval optimizes the pacing interval for a left ventricle.

6. A medium according to claim 1, wherein the physiologic sensor variable includes an autonomically controlled variable and is one of pressure, cardiac output, or cardiac flow.

7. A medium according to claim 6, wherein the autonomically controlled variable includes pressure, and the change in the physiologic sensor variable is a change in pressure.

8. A medium according to claim 1, wherein the transient change in the value of the physiologic sensor variable forms a slope of an initial deflection from the baseline physiologic sensor variable value.

9. The method of claim 1, further comprising instructions for causing the programmable processor to interpret a transient increase in a physiologic sensor variable as corresponding to a superior pacing interval, a transient decrease in a physiologic sensor variable as corresponding to an inferior pacing interval, and an absence of transient change in a physiologic sensor variable as corresponding to an equivalent pacing interval relative to the baseline pacing interval.

10. A computer-readable medium programmed with instructions for performing a method of optimization of a pacing interval in an implantable medical device, the medium comprising instructions for causing a programmable processor to:
set the pacing interval to a baseline;
measure a value of baseline physiologic sensor variable at the baseline pacing interval;
iteratively change the pacing interval setting two or more times, to measure the transient change in the value of the physiologic sensor variable in response to the change in the pacing interval setting at each pacing interval setting;
change the pacing interval setting based on at least one measured value of the transient change;
determine a maximum transient change of the measured transient changes; and
identify the pacing interval associated with the maximum transient change in the sensor variable as an optimal pacing interval.

11. A medium according to claim 10, wherein the physiologic sensor variable measurement utilizes an implanted sensor.

12. A medium according to claim 10, wherein the sensor variable includes the RV pressure and the pacing interval includes the AV interval.

13. A medium according to claim 10, wherein the measurement of the baseline physiologic sensor variable occurs at an elevated heart rate of approximately 10 to 40 beats per minute above a resting heart rate.

14. A medium according to claim 10, wherein the optimization process includes repeated evaluation at more than one elevated heart rate.

15. A medium according to claim 14, wherein the optimal pacing intervals identified at the more than one elevated heart rate are used to determine the optimal pacing intervals for a range of heart rates.

16. A medium according to claim 10, wherein each change to the pacing interval setting is from the baseline pacing interval.

17. A computer-readable medium programmed with instructions for performing a method of optimization of a pacing interval in an implantable medical device, the medium comprising instructions for causing a programmable processor to:
set the pacing interval to a baseline; measure a value of baseline physiologic sensor variable at the baseline pacing interval;
change the pacing interval setting two or more times;
measure the transient change in the value of the physiologic sensor variable in response to the change in the pacing interval setting at each pacing interval setting;
determine a maximum transient change of the measured transient changes, the maximum transient change being determined by fitting a curve to the measured transient changes to determine the point on the curve where the change is maximum; and
identify the pacing interval associated with the maximum transient change in the sensor variable as an optimal pacing interval.

18. A medium according to claim 17, wherein the change to the pacing interval setting two or more times includes a progressive increase in the pacing interval by discrete steps.

19. A medium according to claim 17, wherein the change to the pacing interval setting two or more times occurs until the maximum transient change of the measured transient changes is determined.

20. A medium according to claim 17, wherein the measurement of the transient change in the value of the physiologic sensor variable for each pacing interval setting occurs over several cardiac cycles.

21. An apparatus adapted for optimizing a pacing interval in an implantable medical device, comprising:
means for setting a pacing interval to a baseline pacing interval;
means for measuring a value of a baseline physiologic sensor variable at the baseline pacing interval;
means for changing the base line pacing interval setting at least two more times;
means for measuring a temporary excursion in the value of the physiologic sensor variable in response to the change in the pacing interval setting at each pacing interval setting;
means for determining a maximum transient change of the measured temporary excursions;
means for identifying a pacing interval associated with the maximum transient change in the sensor variable; and
means for programming the pacing interval for optimized therapy delivery by adjusting the pacing interval by at least one physiologic offset interval.
